(19) United States
(12) Patent Application Publication
(10) Pub. No.: US 2025/0340947 A1
(43) Pub. Date: Nov. 6, 2025

(54) EARLY DETECTION OF CANCER USING ALLELE DRIFT AND CHROMOSOMAL INSTABILITY

(71) Applicant: ROSWELL PARK CANCER INSTITUTE CORPORATION, Buffalo, NY (US)

(72) Inventors: Carl MORRISON, Buffalo, NY (US); Sean GLENN, Buffalo, NY (US); Manuel GLYNIAS, Buffalo, NY (US); Phillips GALBO, Buffalo, NY (US); Prashant SINGH, Buffalo, NY (US)

(21) Appl. No.: 18/865,990

(22) PCT Filed: May 16, 2023
(86) PCT No.: PCT/US2023/022361

§ 371 Date: Nov. 14, 2024

Related U.S. Application Data
(60) Provisional application No. 63/342,374, filed on May 16, 2022.
(60) Provisional application No. 63/430,483, filed on Dec. 6, 2022.

(51) Int. Cl. C12Q 1/6886

(52) U.S. Cl. CPC C12Q 1/6886; C12Q 2600/106; C12Q 2600/156

(57) ABSTRACT

The present disclosure is directed generally to methods for characterizing response to one or more therapies in patients with clinically apparent cancer, or recurrence of disease in treated cancer patients with no clinically apparent cancer. Also disclosed are methods for detecting and/or treating a cancer using allele imbalance and chromosomal instability.

### I. CROSS REFERENCE TO RELATED APPLICATIONS

This application claims the benefit of U.S. Provisional Application No. 63/342,374, filed on May 16, 2022, and 63/430,483, filed on Dec. 6, 2022, applications which are incorporated herein by reference in their entirety.

### II. BACKGROUND

There are currently many methods of characterizing response to one or more therapies in patients with clinically apparent cancer, or recurrence of disease in treated cancer patients with no clinically apparent cancer, i.e., minimal residual disease (MRD). These techniques perform mutational analysis of nonpolymorphic loci. However, response to therapy or MRD using this prior art of measuring DNA mutations at nonpolymorphic loci in a liquid biopsy lacks sensitivity and specificity. What are needed are new methods of detecting cancers and characterizing response to one or more therapies that do not suffer from these limitations.

### III. SUMMARY

Disclosed are methods for the detection of cancer and the characterization of responses to one or more therapies in patients and assessing the efficacy of a treatment regimen.

In one aspect, disclosed herein are methods for detecting a cancer, cancer recurrence, and/or metastasis (including, but not limited to cancer recurrence in a subject previously treated for cancer with no clinically apparent cancer, i.e., MRD) in a subject comprising: a) obtaining a first tissue sample (such as, for example a liquid biopsy including, but not limited to a liquid biopsy comprising whole blood, peripheral blood, plasma, serum, saliva, sputum, cerebral spinal fluid, urine, or lymph) from a subject at a first timepoint; wherein the first time point occurs following treatment for a cancer; b) isolating cell free (cf) deoxyribonucleic acid (DNA) (cfDNA) from the first tissue sample; c) measuring regions of chromosomal homozygosity of polymorphic sites in the first tissue sample (including, but not limited to measuring by next generation sequencing (NGS), allelic-specific hybridization, primer extension, oligonucleotide ligation, and/or invasive cleavage) to obtain an allele ratio thereby creating an internal reference measurement; d) obtaining a second tissue sample from the subject at a second timepoint; wherein the second time point occurs after the first time point; e) isolating cell free cfDNA from the second tissue sample; and f) measuring regions of chromosomal homozygosity change of polymorphic sites in the second tissue sample relative to the measurements obtain with the first tissue sample; wherein when the first tissue sample is obtained following treatment for a cancer, a change in heterozygosity of the cfDNA in the second tissue sample away from the relative heterozygosity of the cfDNA in the first tissue sample indicates the presence of contaminating circulating tumor (ct) DNA (ctDNA) and therefore the presence of a recurrent cancer and/or metastasis; and wherein no change in the heterozygosity of the cfDNA between the first and second tissue samples indicates no recurrent cancer and/or metastasis.

Also disclosed herein are methods for detecting a cancer, cancer recurrence, and/or metastasis of any preceding aspect, wherein the cfDNA in the first or second tissue sample comprises circulating tumor (ct) DNA (ctDNA).

In one aspect disclosed herein are methods for detecting a cancer, cancer recurrence, and/or metastasis of any preceding aspect, further comprising administering to the subject an anti-cancer treatment when a recurrent cancer and/or metastasis is detected.

Also disclosed herein are methods of treating, inhibiting, reducing, decreasing, ameliorating, and/or preventing a cancer, recurrent cancer, and/or metastasis (including, but not limited to cancer recurrence in a subject previously treated for cancer with no clinically apparent cancer, i.e., MRD) in a subject comprising: a) obtaining a first tissue sample (such as, for example a liquid biopsy including, but not limited to a liquid biopsy comprising whole blood, peripheral blood, plasma, serum, saliva, sputum, cerebral spinal fluid, urine, or lymph) from a subject at a first timepoint; wherein the first time point occurs following treatment for a cancer; b) isolating cell free (cf) deoxyribonucleic acid (DNA) (cfDNA) from the first tissue sample; c) measuring regions of chromosomal homozygosity of polymorphic sites in the first tissue sample (including, but not limited to measuring by next generation sequencing (NGS), allelic-specific hybridization, primer extension, oligonucleotide ligation, and/or invasive cleavage) thereby creating an internal reference measurement; d) obtaining a second tissue sample from the subject at a second timepoint; wherein the second time point occurs after the first time point; e) isolating cell free cfDNA from the second tissue sample; f) measuring regions of chromosomal homozygosity change of polymorphic sites in the second tissue sample relative to the measurements obtain with the first tissue sample; wherein a change in heterozygosity of the cfDNA in the second tissue sample away from the relative heterozygosity of the cfDNA in the first tissue sample indicates the presence of contaminating circulating tumor (ct) DNA (ctDNA) and therefore the presence of a recurrent cancer and/or metastasis; and wherein no change in the heterozygosity of the cfDNA between the first and second tissue samples indicates no recurrent cancer and/or metastasis; and administering to the subject an anti-cancer therapeutic when a recurrent cancer and/or metastasis is detected.

In one aspect, disclosed herein are methods of treating, inhibiting, decreasing, reducing, ameliorating, and/or preventing a cancer, recurrent cancer, and/or metastasis (including, but not limited to cancer recurrence in a subject previously treated for cancer with no clinically apparent cancer, i.e., MRD) of any preceding aspect, wherein the cfDNA in the first or second tissue sample comprises circulating tumor (ct) DNA (ctDNA).

Also disclosed are methods of assessing the efficacy a therapeutic regimen to a cancer and/or metastasis (including, but not limited to cancer recurrence in a subject previously treated for cancer with no clinically apparent cancer, i.e., MRD) in a subject comprising: a) obtaining a first tissue sample (such as, for example a liquid biopsy including, but not limited to a liquid biopsy comprising whole blood, peripheral blood, plasma, serum, saliva, sputum, cerebral spinal fluid, urine, or lymph) from a subject at a first timepoint; wherein the first timepoint occurs before treatment with the therapeutic regimen; b) isolating cell free (cf) deoxyribonucleic acid (DNA) (cfDNA) from the first tissue sample; c) measuring regions of chromosomal homozygosity of polymorphic sites in the first tissue sample (including, but not limited to measuring by next generation sequencing (NGS), allelic-specific hybridization, primer extension, oligonucleotide ligation, and/or invasive cleavage) thereby creating an internal reference measurement; d) obtaining a second tissue sample from the subject at a second timepoint; e) isolating cell free cfDNA from the second tissue sample; and f) measuring regions of chromosomal homozygosity change of polymorphic sites in the second tissue sample relative to the measurements obtain with the first tissue sample;

In one aspect, disclosed are methods of assessing the efficacy a therapeutic regimen to a cancer and/or metastasis (including, but not limited to cancer recurrence in a subject previously treated for cancer with no clinically apparent cancer, i.e., MRD) of any preceding aspect wherein no change or a change in heterozygosity towards homozygosity in heterologous alleles of the cfDNA in the second tissue sample relative to the cfDNA in the first tissue sample indicates the presence of contaminating circulating tumor (ct) DNA (ctDNA) and therefore the treatment regimen is not efficacious; and wherein a change in heterozygosity towards heterozygosity in heterologous alleles of the cfDNA in the second tissue sample relative to the cfDNA in the first tissue sample indicates the a decrease or absence in contaminating circulating tumor (ct) DNA (ctDNA) and therefore the treatment regimen is efficacious.

Also disclosed herein are methods of assessing the efficacy of a therapeutic regimen to a cancer and/or metastasis (including, but not limited to cancer recurrence in a subject previously treated for cancer with no clinically apparent cancer, i.e., MRD) of any preceding aspect, wherein the cfDNA in the first or second tissue sample comprises circulating tumor (ct) DNA (ctDNA).

In one aspect, disclosed herein are methods of assessing the efficacy of a therapeutic regimen to a cancer and/or metastasis (including, but not limited to cancer recurrence in a subject previously treated for cancer with no clinically apparent cancer, i.e., MRD) of any preceding aspect, further comprising administering to the subject an anti-cancer treatment when a recurrent cancer and/or metastasis is detected.

### IV. BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 shows a representation of 27 base pairs of DNA from white blood cells for both the maternal and paternal chromosomes as represented by the positive strand, of a double stranded helix. Of the 27 base pairs of DNA a total of 7 are known potential polymorphic sites, i.e. single nucleotide polymorphisms (SNPs).

FIG. 2 shows the theoretical results of a A/B allele ratio for the 7 are known potential polymorphic loci previously illustrated in FIG. 1 for cell free DNA (cfDNA) with no contamination by neoplastic cell DNA (ctDNA).

FIG. 3 shows the measured results of a A/B allele ratio for the 7 known potential polymorphic loci previously illustrated in FIG. 1 for cell free DNA (cfDNA) with no contamination by neoplastic cell DNA (ctDNA).

FIG. 4 shows the results of a A/B allele ratio for the 7 known potential polymorphic loci previously illustrated in FIG. 1 for cell free DNA (cfDNA) with no contamination by neoplastic cell DNA (ctDNA).

FIG. 5 shows a representation of neoplastic tissue that has lost one of two chromosomes.

FIG. 6 shows the results of a A/B allele ratio for the 7 known potential polymorphic loci previously illustrated in FIG. 1, but in neoplastic tissue that has lost one of two chromosomes as illustrated in FIG. 5, i.e., for cell free DNA (cfDNA) with 100% neoplastic tissue with “loss of heterozygosity” (LOH)

FIG. 7 shows the results of a A/B allele ratio for the 7 known potential polymorphic loci previously illustrated in FIG. 1, but in neoplastic tissue that has lost one of two chromosomes as illustrated in FIG. 5, i.e., cell free DNA (cfDNA) contaminated with neoplastic cell DNA (ctDNA) that have lost either the maternal or paternal chromosome (i.e. LOH).

FIG. 8 shows a representation of where the paternal copy of one chromosome is lost.

FIG. 9 shows the results of a A/B allele ratio for the 7 known potential polymorphic loci previously illustrated in FIG. 1 for cell free DNA (cfDNA) contaminated with a high number of neoplastic cell DNA (ctDNA) with known allelic imbalance for a specific region of a chromosome in a patient undergoing treatment and being responsive to treatment.

FIG. 10 shows the results of a A/B allele ratio for the 7 known potential polymorphic loci previously illustrated in FIG. 1 for cell free DNA (cfDNA) contaminated with a high number of neoplastic cell DNA (ctDNA) with known allelic imbalance for a specific region of a chromosome in a patient undergoing treatment, but nonresponsive to treatment.

FIG. 11 shows the results of a A/B allele ratio for the 7 known potential polymorphic loci previously illustrated in FIG. 1 for cell free DNA (cfDNA) contaminated with a very low number of neoplastic cell DNA (ctDNA) with known allelic imbalance for a specific region of a chromosome in a patient post-treatment with significant response to treatment with a follow-up for minimal residual disease (MRD) in a patient with recurrence.

FIG. 12 shows the results of a A/B allele ratio for the 7 known potential polymorphic loci previously illustrated in FIG. 1 for cell free DNA (cfDNA) contaminated with a very low number of neoplastic cell DNA (ctDNA) with known allelic imbalance for a specific region of a chromosome in a patient post-treatment with significant response to treatment with a follow-up for minimal residual disease (MRD) in a patient with without recurrence.

### V. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. DEFINITIONS

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

An “increase” can refer to any change that results in a greater amount of a symptom, disease, composition, condition or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The terms “treat,” “treating,” “treatment,” and grammatical variations thereof as used herein, refer to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. Treatments according to the disclosure may be applied preventively, prophylactically, palliatively, or remedially. Treatments are administered to a subject prior to onset (e.g., before obvious signs of cancer), during early onset (e.g., upon initial signs and symptoms of cancer), or after an established development of cancer. Prophylactic administration can occur for several days to years prior to the manifestation of symptoms of a cancer.

“Biocompatible” generally refers to a material and any metabolites or degradation products thereof that are generally non-toxic to the recipient and do not cause significant adverse effects to the subject.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

A “control” is an alternative subject or sample used in an experiment for comparison purposes. A control can be “positive” or “negative.”

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

A “pharmaceutically acceptable” component can refer to a component that is not biologically or otherwise undesirable, i.e., the component may be incorporated into a pharmaceutical formulation provided by the disclosure and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. METHOD OF DETECTING AND TREATING CANCER

There are currently many methods of characterizing response to one or more therapies in patients with clinically apparent cancer, or recurrence of disease in treated cancer patients with no clinically apparent cancer, i.e., minimal residual disease (MRD). Most recently, the technological advance of next generation sequencing (NGS) has created a prior state of the art in this field. The prior state of the art using NGS requires (i) obtaining tumor sample from the patient, (ii) measuring DNA mutations at nonpolymorphic loci in the tumor, (iii) obtaining future blood samples from the patient during treatment for evaluating response to therapy, or post-treatment for evaluating recurrence of tumor after treatment (iv) isolating cell free DNA (cfDNA) from blood samples (liquid biopsy) that likely contains some level of circulating tumor cell free DNA (catena), and (v) predicting from these cfDNA samples, based upon mutational analysis of nonpolymorphic loci in DNA coding regions, response of the tumor to treatment or recurrence of tumor after treatment.

However, response to therapy or MRD using this prior art of measuring DNA mutations at nonpolymorphic loci in a liquid biopsy lacks sensitivity and specificity. Lack of specificity is primarily due to inherent errors in NGS that impact the probability of a true event at a single nonpolymorphic loci. Although prior methodologies have improved errors in NGS, the prior art of measuring DNA mutations at nonpolymorphic loci still has significant problems with the probability of a true event at low levels of ctDNA. The lack of sensitivity of the prior art of analysis of nonpolymorphic loci to detect response to therapy or MRD relies on the probability of distinguishing multiple mutational independent events from sequencing errors from true changes whereby ctDNA is present in cfDNA. These multiple mutational events have no outcome in common and are called disjoint in regard to the probability of detecting a true event. For the prior art the basic rule of probability is if two events A and B are disjoint, then the probability of either event is the sum of the probabilities of the two events: P(A or B)=P(A)+P(B). The result of this regarding sensitivity is that up to one-fourth of all true events are not detected. Regarding specificity, the prior art of measuring DNA mutations at nonpolymorphic loci in a liquid biopsy is complicated by clonal hematopoiesis. The latter is the presence of mutations in cells in the blood (i.e., white blood cells) that release DNA into the cfDNA component of a liquid biopsy. Clonal hematopoiesis occurs in a large percentage of older patients with no clinical evidence of cancer. Although prior methodologies have improved the distinction of mutations in cfDNA due to clonal hematopoiesis (false positives) versus true positives of tumor recurrence, the results are less than optimal with up to 50% of false positive calls.

The present disclosure is directed to a method for characterizing response to therapy and MRD based upon AI and CIN. All human cells contain 46 chromosomes, 23 of maternal and 23 of paternal origin, referred to as autosomal chromosomes 1 through 22 and the sex chromosomes X or Y. When an entire or partial area of a chromosome is lost or gained through cell replication or other processes the loss of the contribution of one parent results in a change of allele balance.

Chromosomes are made up of deoxyribonucleic acid (DNA) and histone proteins with the former being that component that is transmitted from parent to child with rigor. DNA is made up four basic building blocks of adenine (A), cytosine (C), guanine (G), and thymine (T) for which there are approximately 3 billion such blocks across all 46 chromosomes. Since DNA is double stranded with a positive and negative strand there are always 2 of these blocks at each of these 3 billion positions that is referred to as base pairs. Close to 99% of these base pair positions are identical for all humans, referred to as nonpolymorphic loci or alleles, and the remaining 1% of positions that are different in greater than 95% of the population are called polymorphisms, or more specifically single nucleotide polymorphisms (SNPs). There are about 14 million SNPs in our DNA, or roughly one every 200 base pairs across the total 3 billion base pair positions. There is a great deal of variability from one person to the next for specific SNPs in their DNA and can be used as a molecular fingerprint in forensics for the investigation of crimes and other applications. As prior technologies only refer to the plus strand of DNA, for which everyone has two copies of for each chromosome, there are n to the fourth combinations of A, C, G, and T at each polymorphic loci. When both plus strands contain the same DNA molecule such as A and A, C and C, G and G, or T and T, that condition is referred to as homozygous. When both plus strands contain a different DNA molecule such as A and T, A and C, A and G, A and A, C and T, etc. then that condition is referred to as heterozygous with the more common nucleotide in the general population referred to as the “A allele” and the less common as the “B allele”. On any positive strand of DNA with multiple linked and aligned heterozygous loci there can be any combination of “A” and “B” alleles and with the opposite designation on the other minus strand.

Cancers often lose or gain an entire or partial area of a chromosome to be more efficient in cell replication. When this occurs heterozygous SNPs in this area of chromosomal loss or gain, so-called chromosomal instability (CIN), then revert to a state of homozygosity. Just as the presence of SNPs with two matched but different base pairs at given location can be used as a fingerprint in forensics, the comparison of SNPs in matched normal and tumor tissue can be used in a similar manner to investigate the presence or absence of cancer in cfDNA. As change in homozygosity in tumors almost always involved hundreds of thousands to millions of consecutive base pairs of DNA, there can be many unique heterozygous SNPs in that same area that now revert to a homozygous state. These SNPs affected by loss or gain of a specific piece of a chromosome are physically linked and the result is multiple alleles or loci changing from a heterozygous to homozygous state as one event that we refer to as “allele drift”. In a comparable fashion complete or partial loss of a maternal or paternal chromosome in a tumor can result in a copy number change that we refer to as “copy number drift”. As tumors can lose either a maternal or paternal chromosome and then duplicate the remaining copy there are instances where there is copy number change, but there is no copy number drift. In these instances, there will be allele drift when there is no copy number drift and is why the former is a preferred method of analysis for response to therapy or MRD.

As compared to the prior art of looking for change in disjointed nonpolymorphic loci as the sum of independent events to detect cancer in a liquid biopsy our invention allows for the identification of change in multiple joined polymorphic loci or regions of allele drift. When two events are not independent but rather joined as in heterozygous loci in a region of change the result is a conditional probability. If change in one heterozygous loci is detected in a region of change, then it is more likely that change will be detected at a second joined loci, etc. Since events A and B are not independent, then the probability of the intersection of A and B (the probability that both events occur) is defined by P(A and B)=P(A)P(B|A). Given that any region of change will have many joined heterozygous loci the conditional probabilities of any one loci is dependent on the consideration of the detection of change at any preceding loci. As compared to the disjointed probability of detecting change in the prior art, with this methodology in this embodiment the likelihood of detecting contamination in cfDNA with neoplastic cells (ctDNA) is based upon the conditional probability of detecting change at multiple adjacent heterozygous loci in a region of known change, i.e. allele drift.

In one aspect, disclosed herein are methods for detecting a cancer, cancer recurrence, and/or metastasis (including, but not limited to cancer recurrence in a subject previously treated for cancer with no clinically apparent cancer, i.e., MRD) in a subject comprising: a) obtaining a first tissue sample (such as, for example a liquid biopsy including, but not limited to a liquid biopsy comprising whole blood, peripheral blood, plasma, serum, saliva, sputum, cerebral spinal fluid, urine, or lymph) from a subject at a first timepoint; wherein the first time point occurs following treatment for a cancer; b) isolating cell free (cf) deoxyribonucleic acid (DNA) (cfDNA) from the first tissue sample; c) measuring regions of chromosomal homozygosity of polymorphic sites in the first tissue sample (including, but not limited to measuring by next generation sequencing (NGS), allelic-specific hybridization, primer extension, oligonucleotide ligation, and/or invasive cleavage) to obtain an allele ratio thereby creating an internal reference measurement; d) obtaining a second tissue sample from the subject at a second timepoint; wherein the second time point occurs after the first time point; e) isolating cell free cfDNA from the second tissue sample; and f) measuring regions of chromosomal homozygosity change of polymorphic sites in the second tissue sample relative to the measurements obtain with the first tissue sample; wherein when the first tissue sample is obtained following treatment for a cancer, a change in heterozygosity of the cfDNA in the second tissue sample away from the relative heterozygosity of the cfDNA in the first tissue sample indicates the presence of contaminating circulating tumor (ct) DNA (ctDNA) and therefore the presence of a recurrent cancer and/or metastasis; and wherein no change in the heterozygosity of the cfDNA between the first and second tissue samples indicates no recurrent cancer and/or metastasis.

The disclosed methods for detecting a cancer, cancer recurrence, or metastasis (including, but not limited to cancer recurrence in a subject previously treated for cancer with no clinically apparent cancer, i.e., MRD) were developed in recognition that circulating tumor DNA can be present in in cfDNA samples where cancer remains, a recurrent cancer is developing or has formed, and/or a metastasis has occurred. Thus, in one aspect, disclosed herein are methods for detecting a cancer, wherein the cfDNA in the first or second tissue sample comprises circulating tumor (ct) DNA (ctDNA).

Detecting and/or measuring homozygosity/heterozygosity via allelic imbalance, and/or chromosomal instability at polymorphic sites in the tissue samples can be obtained by any means known in the art, including, but not limited to next generation sequencing (NGS), allelic-specific hybridization, primer extension, oligonucleotide ligation, and/or invasive cleavage.

A custom chromosomal instability (CIN) NGS caller was developed to identify concordance of tumor and cfDNA samples. Prior to analysis a quality control (QC) at the individual SNP level for each subject is performed. The total number of reads for all polymorphic (informative) SNPs across all somatic chromosomes in tumor and cfDNA must sum to 50 or greater to pass for further analysis. For those qualified SNPs the frequency of each qualified A and B allele for polymorphic (informative) SNPs is used to calculate the A/total allele ratio in an individual's PBMC sample. Values for the PBMC A/total allele ratio greater than 0.75 or less than 0.25 are considered polymorphisms:

$$1 ) A : {totalallele}_{PBMC} \geq 0.75 \vert \leq 0.25 = polymorphic SNP$$

The absolute SNP difference for each polymorphic SNP using A/total allele ratio for PBMC to tumor is calculated and recorded as CIN SNP difference for each individual SNP:

$$2 ) A : {totalalleles}_{PBMC} \vert - \vert A : {totalalleles}_{TUMOR} = {Δ}_{SNP}$$

Normal chromosomes are identified using a percentage of SNP differences that are greater than a threshold of 0.1 based upon a minimum of 20% neoplastic content for a presumed diploid state. Far extremes of total chromosomal allelic imbalance (AI) versus normal chromosome were identified using percent SNP differences for that chromosome. If 90% or greater of all SNP differences for any chromosome are greater than the threshold of 0.1 then this is considered as whole chromosome AI. Where n=polymorphic SNP and k=threshold Δ_{SNP}:

$$3 ) {\sum }_{k > 0.1}^{n} \geq 90 \% ⇒ {WholeChr}_{AI}$$

If 10% or less of all SNP differences for any chromosome were not greater than 0.1 then this was considered as a normal chromosome with no AI:

$$4 ) {\sum }_{k \geq 01}^{n} \leq 10 \% ⇒ NormChr$$

To identify partial chromosome AI, divide all SNPs into consecutive groups of 50 (i.e. SNP-group) for each autosomal chromosome. Among groups of 50 SNPs calculate percentage above or below baseline threshold of B allele frequency determined by healthy controls. If 70% of a group of 50 SNPs is above or below baseline threshold of B allele frequency determined by healthy controls, then group is considered candidate for partial chromosome AI. To be classified as partial chromosome AI two or more consecutive SNP-groups must be above or below baseline threshold of B allele frequency determined by healthy controls.

Further analysis at this point requires identification of breakpoints in chromosomes with partial AI and excludes those with whole chromosomal AI and normal chromosomes. Breakpoints are identified by finding 10 or more consecutive (by nucleotide position) polymorphic SNPs starting at the p-terminus with a SNP difference greater than 1.5× of SD of the absolute SNP differences for normal chromosomes for that case. Once a breakpoint is identified that defines the beginning of a region of AI for that specific chromosome. Termination of a region of AI requires the encounter of one absolute SNP difference less than 1.5× of SD of the absolute SNP differences for normal chromosomes for that case. Where n=polymorphic SNP and j is standard deviation of NormChr:

$$5 ) {PartialChr}_{AI} {\sum }_{k \geq 1.5 * j}^{n} ∈ {Δ}_{SNP} n + 1 … n + 10 …∩ n < k ⇒ {Region}_{AI} ,$$

$$6 ) {PartialChr}_{AI} - {Region}_{AI} ⇒ {No}_{AI}$$

After identifying whole and partial chromosomal changes allele with greater count is referred to as the retained allele (RA) for each SNP and the lesser as the lost allele (LA). A tumor retained allele fraction (tRAF) is then calculated for each SNP by dividing the tumor retained allele count by the tumor lost allele count:

$$7 ) tRAF = tRAC / tLAC$$

RA and LA designations from the tumor are then used to classify each SNP in the germline as retained or lost. A germline retained allele fraction (gRAF) is then calculated for each SNP by dividing the germline retained allele count (gRAC) by the germline lost allele count (gLAC):

$$8 ) gRAF = gRAC / gLAC$$

A CIN score for the tumor (tCIN) is then calculated using the difference of sum of tRAF and gRAF in a region of CIN:

$$9 ) tCIN score = sum ( tRAF ) - sum ( gRAF )$$

RA and LA designations from the tumor are then used to classify each SNP in the cfDNA as retained or lost. A cfDNA retained allele fraction (cfRAF) is then calculated for each SNP by dividing the cfDNA retained allele count (cfRAC) by the cfDNA lost allele count (cfLAC):

$$10 ) cfRAF = cfRAC / cfLAC$$

A CIN score for the subject cfDNA (cfCIN) is then calculated using the difference of sum of cfRAF and gRAF in a region of CIN in an individual with or prior evidence of cancer.

$$11 ) cfCIN score = sum ( cfRAF ) - sum ( gRAF )$$

A CIN score for the healthy control cfDNA (HCcfCIN) is then calculated using the difference of sum of cfRAF and gRAF in a region of CIN taken from a combined group of subjects with no evidence of cancer.

$$12 ) HCcfCIN score = sum ( HCcfRAF ) - sum ( HCgRAF )$$

Identification of circulating tumor DNA in subject cfDNA, or positive score, requires cfCIN to be greater than HCcfCIN.

$$13 ) Positive ctCIN score = cfCIN > HCcfCIN$$

Also disclosed are methods of assessing the efficacy a therapeutic regimen to a cancer and/or metastasis (such as, for example, the need for continued or renewed treatment to inhibit, reduce, decrease, treat, ameliorate, and/or prevent cancer recurrence (including, but not limited to cancer recurrence in a subject previously treated for cancer with no clinically apparent cancer, i.e., MRD)) in a subject previously treated for cancer with no clinically apparent cancer, i.e., MRD) in a subject comprising: a) obtaining a first tissue sample (such as, for example a liquid biopsy including, but not limited to a liquid biopsy comprising whole blood, peripheral blood, plasma, serum, saliva, sputum, cerebral spinal fluid, urine, or lymph) from a subject at a first timepoint; wherein the first timepoint occurs before treatment with the therapeutic regimen; b) isolating cell free (cf) deoxyribonucleic acid (DNA) (cfDNA) from the first tissue sample; c) measuring regions of chromosomal homozygosity of polymorphic sites in the first tissue sample (including, but not limited to measuring by next generation sequencing (NGS), allelic-specific hybridization, primer extension, oligonucleotide ligation, and/or invasive cleavage) thereby creating an internal reference measurement; d) obtaining a second tissue sample from the subject at a second timepoint; e) isolating cell free cfDNA from the second tissue sample; and f) measuring regions of chromosomal homozygosity change of polymorphic sites in the second tissue sample relative to the measurements obtain with the first tissue sample; wherein no change or a change in heterozygosity towards homozygosity in heterologous alleles of the cfDNA in the second tissue sample relative to the cfDNA in the first tissue sample indicates the presence of contaminating circulating tumor (ct) DNA (ctDNA) and therefore the treatment regimen is not efficacious; and wherein a change in heterozygosity towards heterozygosity in heterologous alleles of the cfDNA in the second tissue sample relative to the cfDNA in the first tissue sample indicates the a decrease or absence in contaminating circulating tumor (ct) DNA (ctDNA) and therefore the treatment regimen is efficacious.

In one aspect, disclosed herein are methods of assessing the efficacy of a therapeutic regimen to a cancer and/or metastasis (such as, for example, the need for continued or renewed treatment to inhibit, reduce, decrease, treat, ameliorate, and/or prevent cancer recurrence (including, but not limited to cancer recurrence in a subject previously treated for cancer with no clinically apparent cancer, i.e., MRD)), wherein the cfDNA in the first or second tissue sample comprises circulating tumor (ct) DNA (ctDNA).

It is understood and herein contemplated that where a recurrent cancer or metastasis is detected using any of the methods disclosed herein or the efficacy of a therapeutic regimen is found wanting, it is desirous to treat the recurrent cancer and/or metastasis. Thus, in one aspect disclosed herein are methods for detecting a cancer recurrence or metastasis or methods of assessing the efficacy of a therapeutic regimen to a cancer and/or metastasis (such as, for example, the need for continued or renewed treatment to inhibit, reduce, decrease, treat, ameliorate, and/or prevent cancer recurrence (including, but not limited to cancer recurrence in a subject previously treated for cancer with no clinically apparent cancer, i.e., MRD)), further comprising administering to the subject an anti-cancer treatment when a recurrent cancer and/or metastasis is detected.

The disclosed compositions can be used to treat any disease where uncontrolled cellular proliferation occurs such as cancers. A representative but non-limiting list of cancers that the disclosed compositions can be used to treat is the following: lymphomas such as B cell lymphoma and T cell lymphoma; mycosis fungoides; Hodgkin's Disease; myeloid leukemia (including, but not limited to acute myeloid leukemia (AML) and/or chronic myeloid leukemia (CML)); bladder cancer; brain cancer; nervous system cancer; head and neck cancer; squamous cell carcinoma of head and neck; renal cancer; lung cancers such as small cell lung cancer, non-small cell lung carcinoma (NSCLC), lung squamous cell carcinoma (LUSC), and Lung Adenocarcinomas (LUAD); neuroblastoma/glioblastoma; ovarian cancer; pancreatic cancer; prostate cancer; skin cancer; hepatic cancer; melanoma; squamous cell carcinomas of the mouth, throat, larynx, and lung; cervical cancer; cervical carcinoma; breast cancer including, but not limited to triple negative breast cancer; genitourinary cancer; pulmonary cancer; esophageal carcinoma; head and neck carcinoma; large bowel cancer; hematopoietic cancers; testicular cancer; and colon and rectal cancers.

Also disclosed herein are methods of treating, inhibiting, decreasing, reducing, ameliorating, and/or preventing a cancer, cancer recurrence, and/or metastasis (including, but not limited to cancer recurrence in a subject previously treated for cancer with no clinically apparent cancer, i.e., MRD)) in a subject comprising: a) obtaining a first tissue sample (such as, for example a liquid biopsy including, but not limited to a liquid biopsy comprising whole blood, peripheral blood, plasma, serum, saliva, sputum, cerebral spinal fluid, urine, or lymph) from a subject at a first timepoint; wherein the first time point occurs following treatment for a cancer; b) isolating cell free (cf) deoxyribonucleic acid (DNA) (cfDNA) from the first tissue sample; c) measuring regions of chromosomal homozygosity of polymorphic sites in the first tissue sample (including, but not limited to measuring by next generation sequencing (NGS), allelic-specific hybridization, primer extension, oligonucleotide ligation, and/or invasive cleavage) thereby creating an internal reference measurement; d) obtaining a second tissue sample from the subject at a second timepoint; wherein the second time point occurs after the first time point; e) isolating cell free cfDNA from the second tissue sample; f) measuring regions of chromosomal homozygosity change of polymorphic sites in the second tissue sample relative to the measurements obtain with the first tissue sample; wherein a change in heterozygosity of the cfDNA in the second tissue sample away from the relative heterozygosity of the cfDNA in the first tissue sample indicates the presence of contaminating circulating tumor (ct) DNA (ctDNA) and therefore the presence of a recurrent cancer and/or metastasis; and wherein no change in the heterozygosity of the cfDNA between the first and second tissue samples indicates no recurrent cancer and/or metastasis; and administering to the subject an anti-cancer therapeutic when a recurrent cancer and/or metastasis is detected.

In one aspect, disclosed herein are methods of treating, inhibiting, decreasing, reducing, ameliorating, and/or preventing a cancer, recurrent cancer, and/or metastasis (including, but not limited to cancer recurrence in a subject previously treated for cancer with no clinically apparent cancer, i.e., MRD)), wherein the cfDNA in the first or second tissue sample comprises circulating tumor (ct) DNA (ctDNA).

It is understood and herein contemplated that the disclosed treatment regimens can used alone or in combination with any anti-cancer therapy known in the art including, but not limited to Abemaciclib, Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin Chlorambucil), Amifostine, Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Atezolizumab, Avastin (Bevacizumab), Avelumab, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin), Bevacizumab, Bexarotene, Bexxar (Tositumomab and Iodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Campath (Alemtuzumab), Camptosar, (Irinotecan. Hydrochloride), Capecitabine, CAPOX, Carac (Fluorouracil—Topical), Carboplatin, CARBOPLATIN-TAXOL, Carfilzomib, Carmubris (Carmustine), Carmustine, Carmustine Implant, Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), Copanlisib Hydrochloride, COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil—Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista, (Raloxifene Hydrochloride), Evomela (Melphalan Hydrochloride), Exemestane, 5-FU (Fluorouracil Injection), 5-FU (Fluorouracil—Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil—Topical), Fluorouracil Injection, Fluorouracil—Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Iodine I 131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and, Hyaluronidase Human, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq, (Atezolizumab), Temodar (Temozolomide), Temozolomide, Temsirolimus, Thalidomide, Thalomid (Thalidomide), Thioguanine, Thiotepa, Tisagenlecleucel, Tolak (Fluorouracil—Topical), Topotecan Hydrochloride, Toremifene, Torisel (Temsirolimus), Tositumomab and Iodine I 131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Vandetanib, VAMP, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), and/or Zytiga (Abiraterone Acetate). The treatment methods can include or further include checkpoint inhibitors including, but are not limited to antibodies that block PD-1 (such as, for example, Nivolumab (BMS-936558 or MDX1106), pembrolizumab, CT-011, MK-3475), PD-L1 (such as, for example, atezolizumab, avelumab, durvalumab, MDX-1105 (BMS-936559), MPDL3280A, or MSB0010718C), PD-L2 (such as, for example, rHIgM12B7), CTLA-4 (such as, for example, Ipilimumab (MDX-010), Tremelimumab (CP-675,206)), IDO, B7-H3 (such as, for example, MGA271, MGD009, omburtamab), B7-H4, B7-H3, T cell immunoreceptor with Ig and ITIM domains (TIGIT) (such as, for example BMS-986207, OMP-313M32, MK-7684, AB-154, ASP-8374, MTIG7192A, or PVSRIPO), CD96, B- and T-lymphocyte attenuator (BTLA), V-domain Ig suppressor of T cell activation (VISTA) (such as, for example, JNJ-61610588, CA-170), TIM3 (such as, for example, TSR-022, MBG453, Sym023, INCAGN2390, LY3321367, BMS-986258, SHR-1702, RO7121661), LAG-3 (such as, for example, BMS-986016, LAG525, MK-4280, REGN3767, TSR-033, BI754111, Sym022, FS118, MGD013, and Immutep).

### C. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1

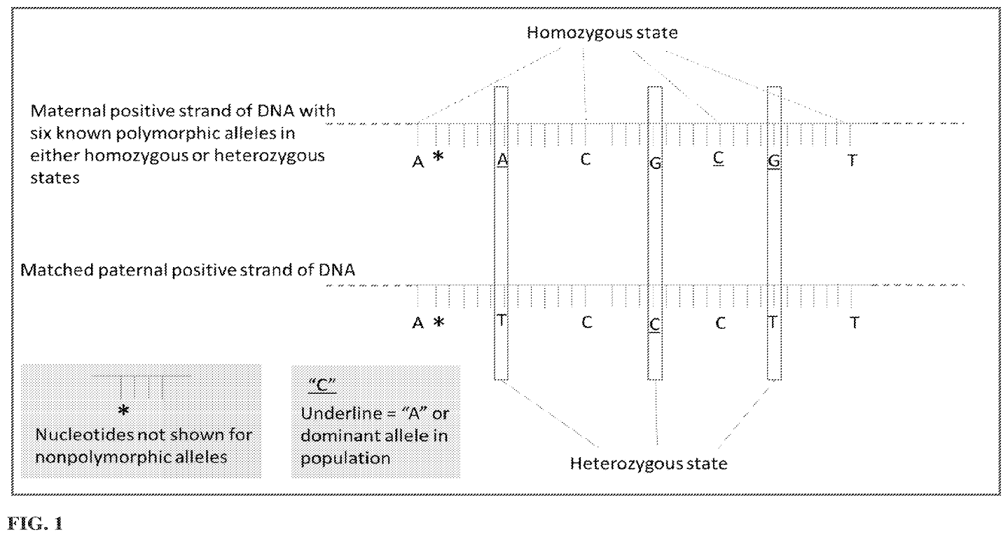

FIG. 1 is an example of 27 base pairs of DNA from white blood cells for both the maternal and paternal chromosomes as represented by one strand, i.e. the positive strand, of a double stranded helix, in accordance with an embodiment. Of the 27 base pairs of DNA a total of 7 are known potential polymorphic sites, or specific nucleotides of DNA that can vary from one person to the next, i.e. single nucleotide polymorphisms (SNPs). When the same nucleotide exists on both the maternal and paternal chromosomes at one of these sites the state is referred to as homozygous. In this example there are 4 potentially polymorphic sites that exist in a homozygous state and for which there is no value for early detection of cancer. Three potential polymorphic sites have a different nucleotide on both the maternal and paternal chromosomes and exist in a state referred to as heterozygous and which have value for early detection of cancer. For those sites in a heterozygous state the specific nucleotide that is more common in the general population is called the “A” allele and can be on either the maternal or paternal chromosome. The less common nucleotide in the general population is called the “B” allele.

### Example 2: Theoretical cfDNA with No ctDNA Contamination

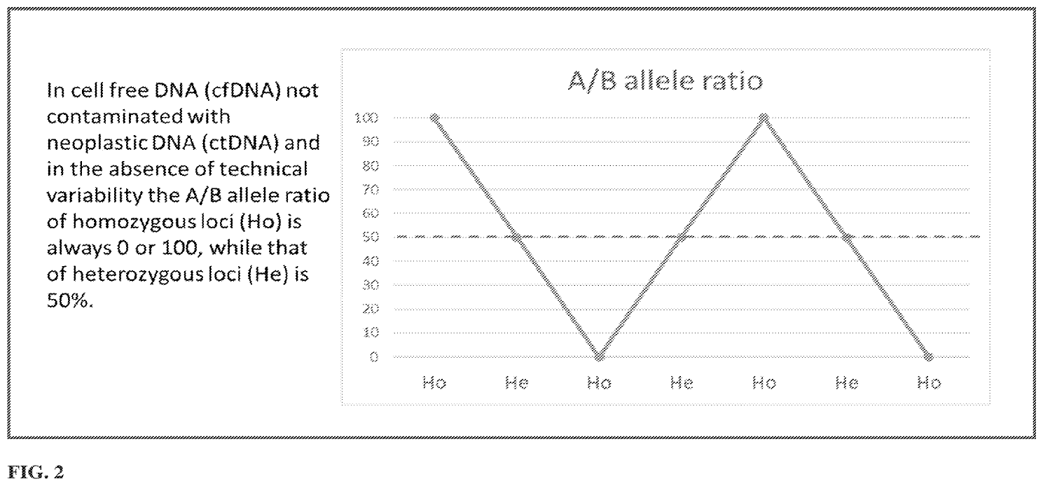

FIG. 2 shows the results of a A/B allele ratio for the 7 are known potential polymorphic loci previously illustrated in FIG. 1 for cell free DNA (cfDNA) with no contamination by neoplastic cell DNA (ctDNA). In this invention the A/B allele ratio is measured by counting the number of times the A allele nucleotide at a specific DNA position is observed in a single next generation sequencing evaluation of a single sample divided by the value for the respective B allele. In this example measurement of the A/B allele ratio for the 4 homozygous polymorphic loci would result in a theoretical value of either 0 or 1. Measurement of the A/B allele ratio for the 3 heterozygous polymorphic loci would result in a theoretical value of 0.50.

### Example 3: Measured cfDNA with No ctDNA Contamination

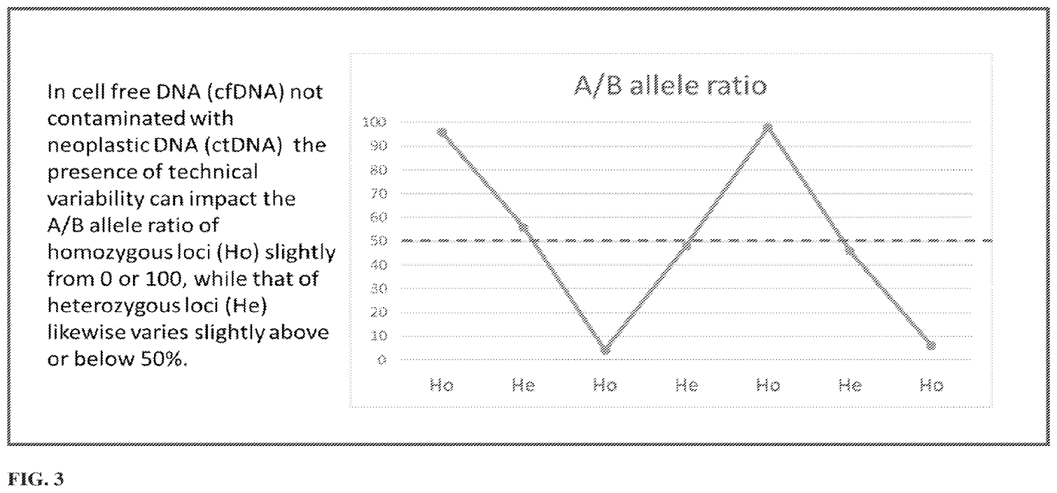

80. FIG. 3 shows the results of a A/B allele ratio for the 7 known potential polymorphic loci previously illustrated in FIG. 1 for cell free DNA (cfDNA) with no contamination by neoplastic cell DNA (ctDNA). The actual measured values for the A/B allele ratios for each loci approximate the theoretical values but are not the same exact value due to technical variability. The degree of technical variability at each polymorphic loci can and does vary per various technologies used to measure allele ratios. This technical variability results in A/B allele ratio values slightly less than 1 or slightly more than 0 for homozygous loci. For heterozygous loci technical variability results in A/B allele ratio values that are slightly greater or less than 0.50.

### Example 4: cfDNA with ctDNA No Contamination

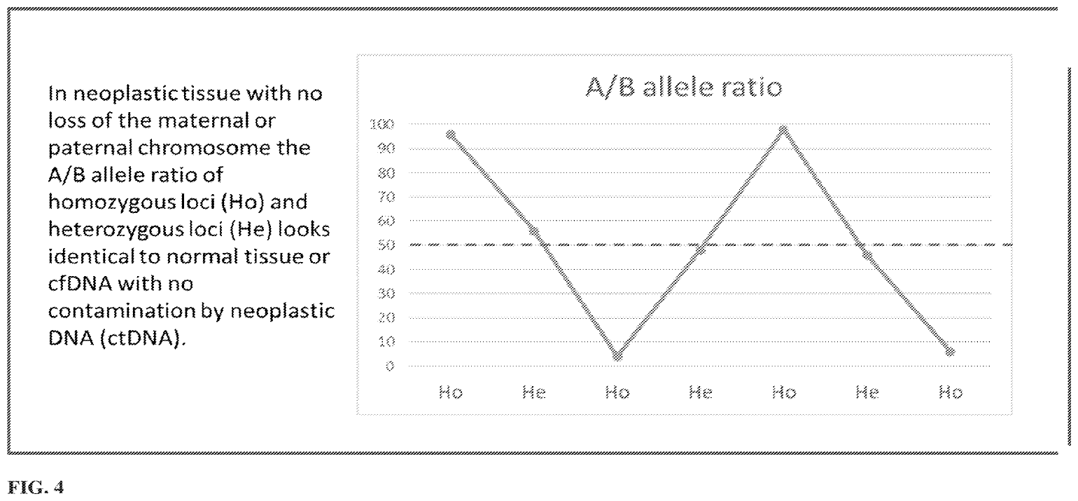

FIG. 4 shows the results of a A/B allele ratio for the 7 known potential polymorphic loci previously illustrated in FIG. 1 for cell free DNA (cfDNA) with no contamination by neoplastic cell DNA (ctDNA). In this example the previous analysis of the neoplastic cells did not show allelic imbalance for these 7 polymorphic loci and there is no impact on the A/B allele ratio values from the theoretical values minus the technical variability.

### Example 5: Loss of Chromosome

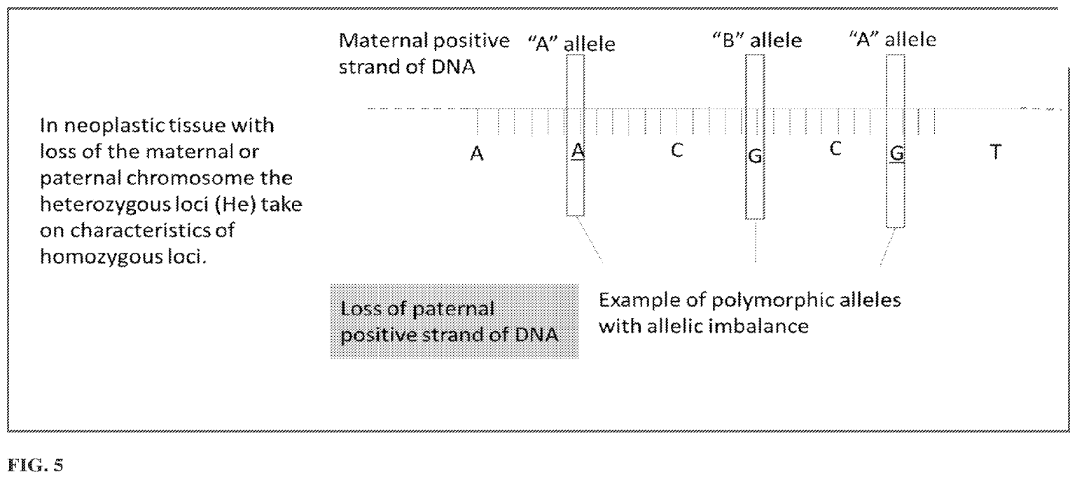

FIG. 5 shows neoplastic tissue that has lost one of two chromosomes, in this instance the paternal copy, and illustrates that alleles that were previously heterozygous now become homozygous.

### Example 6: cfDNA that has Loss of Homozygosity (LOH)

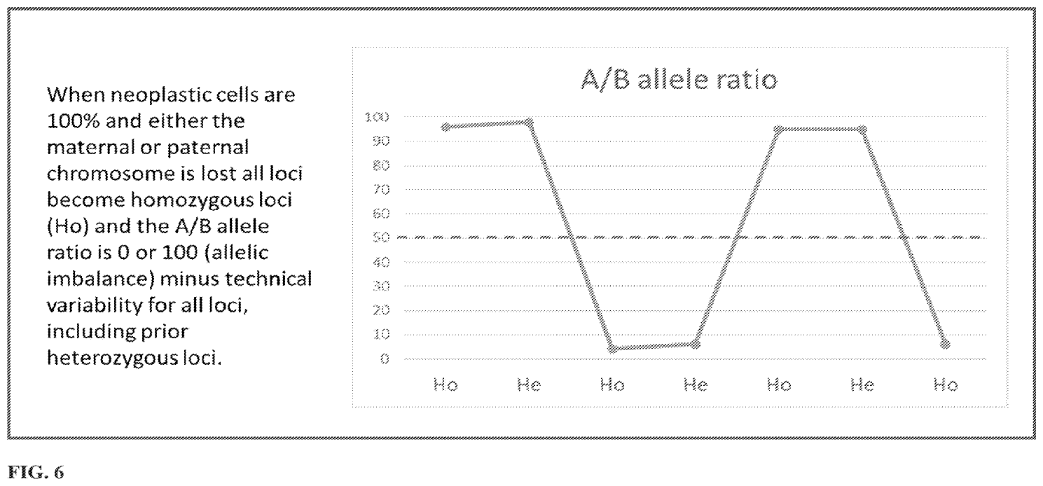

FIG. 6 shows 100% neoplastic tissue with “loss of heterozygosity” (LOH) that results in previously heterozygous loci with an A/B allele ratio equal to 0.50 now having a value of 0 or 1 minus technical variability. The result is all loci now appear to be homozygous.

### Example 7: cfDNA Comprising Contamination with ctDNA that has Loss of Homozygosity (LOH)

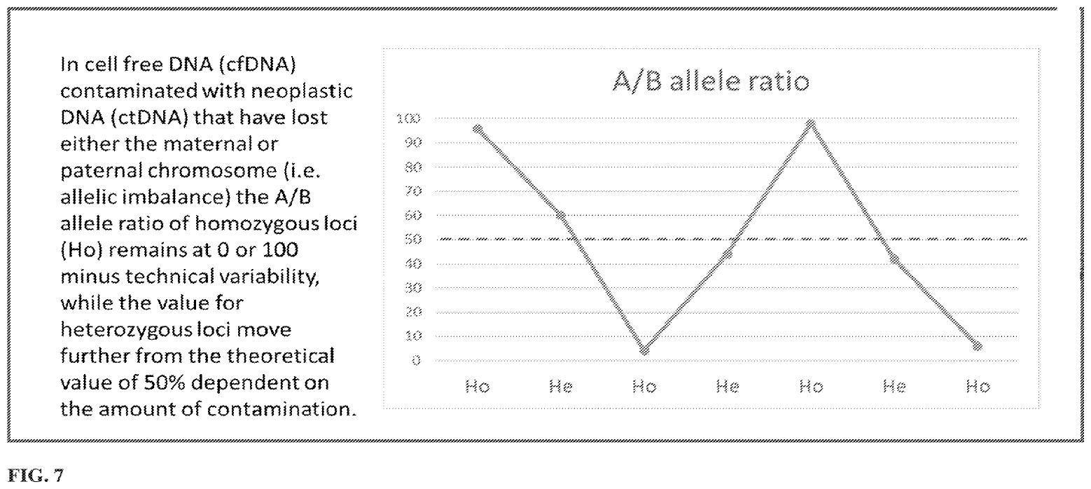

FIG. 7 shows cell free DNA (cfDNA) contaminated with neoplastic cell DNA (ctDNA) that have lost either the maternal or paternal chromosome (i.e. LOH) and the A/B allele ratio of homozygous loci (Ho) remains at 0 or 100 minus technical variability, while the value for heterozygous loci move further from the theoretical value of 50% dependent on the amount of contamination.

### Example 8: Loss of One Copy of a Chromosome

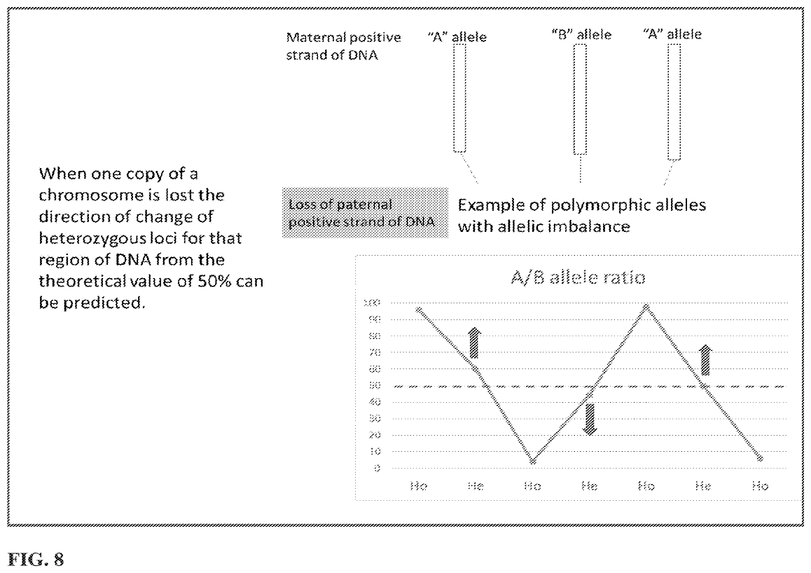

FIG. 8 shows an example where the paternal copy of one chromosome is lost. For this region of DNA, the direction of change of heterozygous loci from the theoretical value of 50% can be predicted after analysis is completed. In this example for the three polymorphic loci from left to right, or 5-prime to 3-prime, the retained alleles are A, B, and A. This results in an increase of the A/B allele ratio when the retained allele is A, and a decrease when the retained allele is B. The increase or decrease of the A/B allele ratio of these loci are joined and move together in a relative fashion, i.e., allele drift, allowing for a conditional probability of detection for this event.

### Example 9: cfDNA Showing Responsive to Treatment

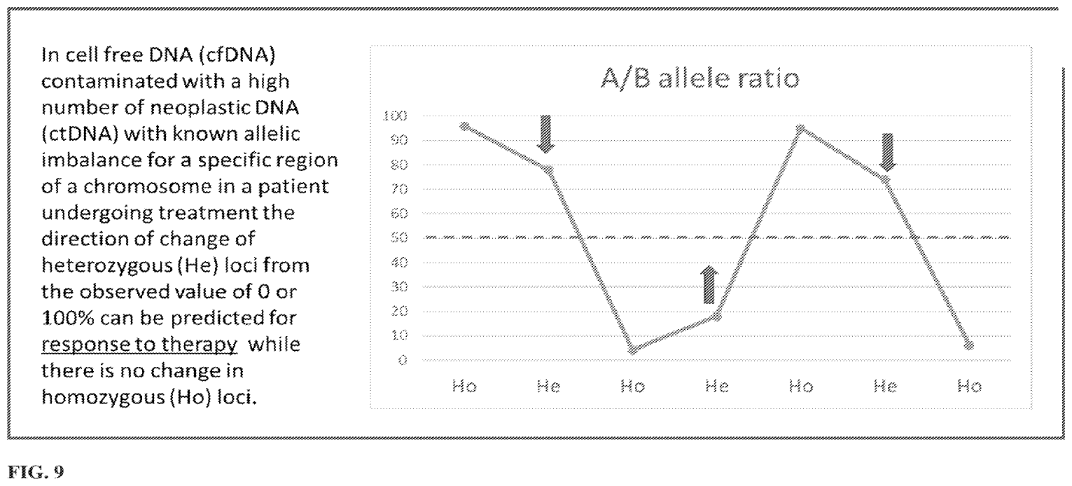

FIG. 9 shows cell free DNA (cfDNA) contaminated with a high number of neoplastic cell DNA (ctDNA) with known allelic imbalance for a specific region of a chromosome in a patient undergoing treatment. In this example three polymorphic loci that are heterozygous in normal cfDNA that would typically have an A/B allele theoretical ratio of 0.50 have values closer to a homozygous loci due to contamination of ctDNA. In this example the three loci have lost from left to right, or 5-prime to 3-prime, the B, A, and B allele. With response to therapy and diminishing ctDNA the direction of change of heterozygous (He) loci from the observed values can be predicted together in a relative fashion, i.e., allele drift, allowing for a conditional probability of detection for this event.

### Example 10: cfDNA that is Non-Responsive to Treatment

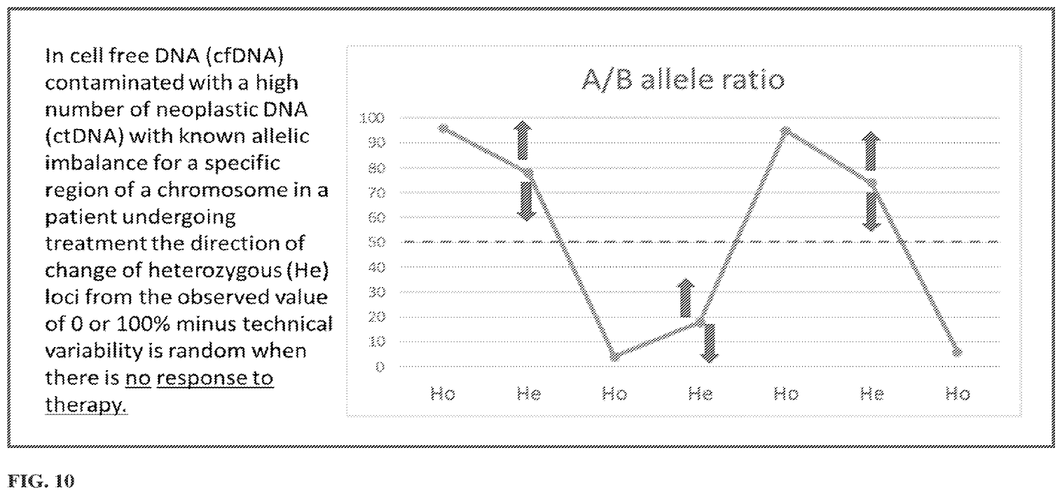

FIG. 10 shows cell free DNA (cfDNA) contaminated with a high number of neoplastic cell DNA (ctDNA) with known allelic imbalance for a specific region of a chromosome in a patient undergoing treatment. In this example three polymorphic loci that are heterozygous in normal cfDNA that would typically have an A/B allele theoretical ratio of 0.50 have values closer to a homozygous loci due to contamination of ctDNA. In this example the three loci have lost from left to right, or 5-prime to 3-prime, the B, A, and B allele. When there is no response to therapy and no decrease in ctDNA the direction of change of heterozygous (He) loci from the observed values cannot be predicted together in a relative fashion, i.e., allele drift, not allowing for a conditional probability of detection for this event.

### Example 11: cfDNA that is Post Treatment with Recurrence

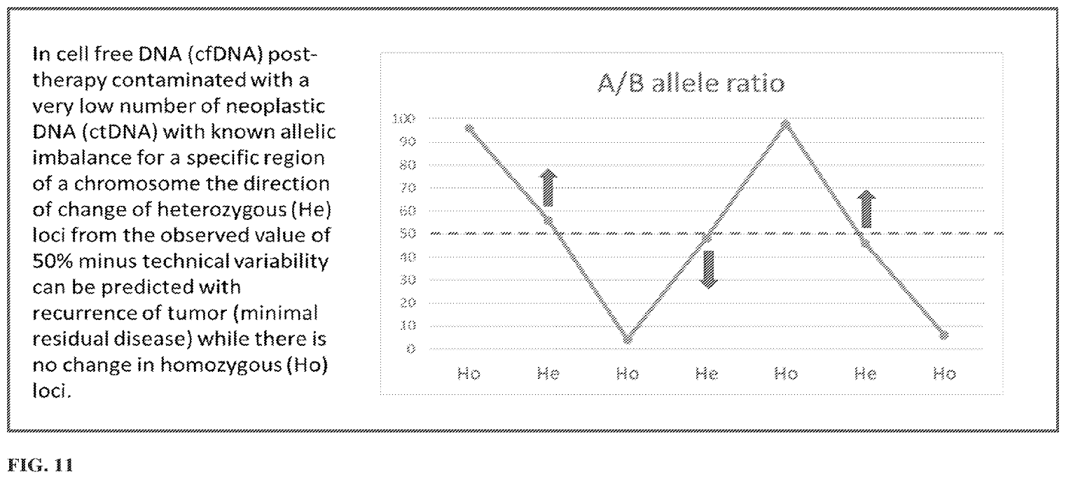

FIG. 11 shows cell free DNA (cfDNA) contaminated with a very low number of neoplastic cell DNA (ctDNA) with known allelic imbalance for a specific region of a chromosome in a patient post-treatment with significant response to treatment. In this example three polymorphic loci that are heterozygous in normal cfDNA have A/B allele ratios close to the theoretical ratio of 0.50 as would be expected with minimal contamination of ctDNA and response to treatment. In this example prior analysis of the tumor showed that the three loci have lost from left to right, or 5-prime to 3-prime, the B, A, and B allele. With follow-up for minimal residual disease (MRD) in a patient with recurrence and increasing ctDNA the direction of change of heterozygous (He) loci from the observed values can be predicted together in a relative fashion, i.e., allele drift, allowing for a conditional probability of detection for this event.

### Example 12: cfDNA that is Post Treatment and No Recurrence

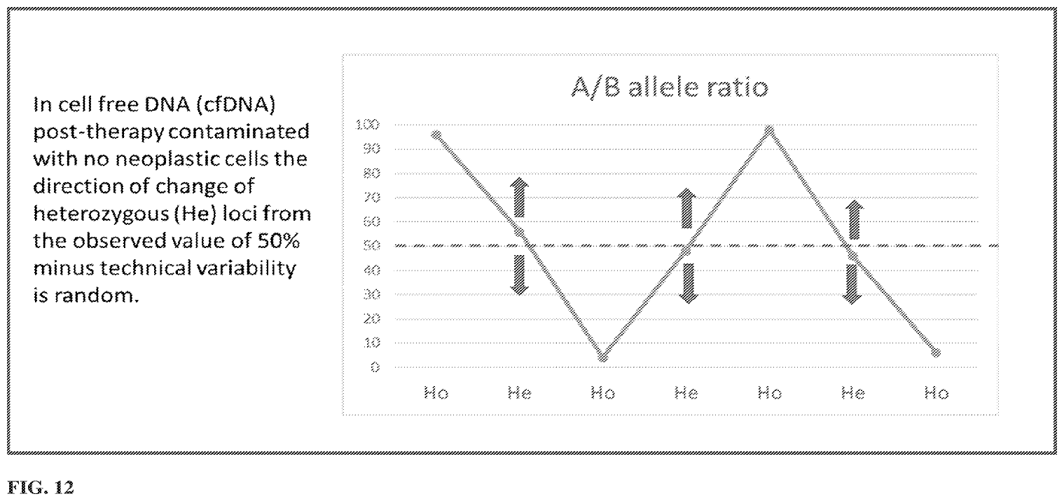

FIG. 12 shows cell free DNA (cfDNA) contaminated with a very low number of neoplastic cell DNA (ctDNA) with known allelic imbalance for a specific region of a chromosome in a patient post-treatment with significant response to treatment. In this example three polymorphic loci that are heterozygous in normal cfDNA have A/B allele ratios close to the theoretical ratio of 0.50 as would be expected with minimal contamination of ctDNA and response to treatment. In this example prior analysis of the tumor showed that the three loci have lost from left to right, or 5-prime to 3-prime, the B, A, and B allele. With follow-up for minimal residual disease (MRD) in a patient without recurrence and no change in ctDNA the direction of change of heterozygous (He) loci from the observed values cannot be predicted together in a relative fashion, i.e., allele drift, not allowing for a conditional probability of detection for this event.

1. A method for detecting a cancer recurrence or metastasis in a subject comprising:
a) obtaining a first tissue sample from a subject at a first timepoint; wherein the first time point occurs following treatment for a cancer;
b) isolating cell free (cf) deoxyribonucleic acid (DNA) (cfDNA) from the first tissue sample;
c) measuring regions of chromosomal homozygosity of polymorphic sites in the first tissue sample to obtain an allele ratio thereby creating an internal reference measurement;
d) obtaining a second tissue sample from the subject at a second timepoint; wherein the second time point occurs after the first time point;
e) isolating cell free cfDNA from the second tissue sample; and
f) measuring regions of chromosomal homozygosity change of polymorphic sites in the second tissue sample relative to the measurements obtain with the first tissue sample;
wherein when the first tissue sample is obtained following treatment for a cancer, a change in heterozygosity of the cfDNA in the second tissue sample away from the relative heterozygosity of the cfDNA in the first tissue sample indicates the presence of contaminating circulating tumor (ct) DNA (ctDNA) and therefore the presence of a recurrent cancer and/or metastasis; and wherein no change in the heterozygosity of the cfDNA between the first and second tissue samples indicates no recurrent cancer and/or metastasis.

2. The method of detecting a recurrent cancer and/or metastasis of claim 1, wherein the tissue sample comprises a liquid biopsy.

3. The method of detecting a recurrent cancer and/or metastasis of claim 2, wherein the tissue obtained in the liquid biopsy comprises whole blood, plasma, serum, saliva, sputum, cerebral spinal fluid, urine, or lymph.

4. The method of detecting a recurrent cancer and/or metastasis of claim 1, wherein the cfDNA in the first or second tissue sample comprises circulating tumor (ct) DNA (ctDNA).

5. The method of detecting a recurrent cancer and/or metastasis of claim 1, wherein chromosomal homozygosity is measured using next generation sequencing (NGS), allelic-specific hybridization, primer extension, oligonucleotide ligation, and/or invasive cleavage.

6. The method of detecting a recurrent cancer and/or metastasis of claim 1, further comprising administering to the subject an anti-cancer treatment when a recurrent cancer and/or metastasis is detected.

7. A method of treating a cancer in a subject comprising:
a) obtaining a first tissue sample from a subject at a first timepoint; wherein the first time point occurs following treatment for a cancer;
b) isolating cell free (cf) deoxyribonucleic acid (DNA) (cfDNA) from the first tissue sample;
c) measuring regions of chromosomal homozygosity of polymorphic sites in the first tissue sample thereby creating an internal reference measurement;
d) obtaining a second tissue sample from the subject at a second timepoint; wherein the second time point occurs after the first time point;
e) isolating cell free cfDNA from the second tissue sample;
f) measuring regions of chromosomal homozygosity change of polymorphic sites in the second tissue sample relative to the measurements obtain with the first tissue sample;
wherein a change in heterozygosity of the cfDNA in the second tissue sample away from the relative heterozygosity of the cfDNA in the first tissue sample indicates the presence of contaminating circulating tumor (ct) DNA (ctDNA) and therefore the presence of a recurrent cancer and/or metastasis; and wherein no change in the heterozygosity of the cfDNA between the first and second tissue samples indicates no recurrent cancer and/or metastasis; and
g) administering to the subject an anti-cancer therapeutic when a recurrent cancer and/or metastasis is detected.

8. The method of treating a recurrent cancer and/or metastasis of claim 7, wherein the tissue sample comprises a liquid biopsy.

9. The method of treating a recurrent cancer and/or metastasis of claim 8, wherein the tissue obtained in the liquid biopsy comprises whole blood, plasma, serum, saliva, sputum, cerebral spinal fluid, urine, or lymph.

10. The method of treating a recurrent cancer and/or metastasis of claim 7, wherein the cfDNA in the first or second tissue sample comprises circulating tumor (ct) DNA (ctDNA).

11. The method of treating a recurrent cancer and/or metastasis of claim 7, wherein chromosomal homozygosity is measured using next generation sequencing (NGS), allelic-specific hybridization, primer extension, oligonucleotide ligation, and/or invasive cleavage.

12. A method of assessing the efficacy a therapeutic regimen to a cancer in a subject comprising:
a) obtaining a first tissue sample from a subject at a first timepoint; wherein the first timepoint occurs before treatment with the therapeutic regimen;
b) isolating cell free (cf) deoxyribonucleic acid (DNA) (cfDNA) from the first tissue sample;
c) measuring regions of chromosomal homozygosity of polymorphic sites in the first tissue sample thereby creating an internal reference measurement;
d) obtaining a second tissue sample from the subject at a second timepoint;
e) isolating cell free cfDNA from the second tissue sample; and
f) measuring regions of chromosomal homozygosity change of polymorphic sites in the second tissue sample relative to the measurements obtain with the first tissue sample;
wherein no change or a change in heterozygosity towards homozygosity in heterologous alleles of the cfDNA in the second tissue sample relative to the cfDNA in the first tissue sample indicates the presence of contaminating circulating tumor (ct) DNA (ctDNA) and therefore the treatment regimen is not efficacious; and wherein a change in heterozygosity towards heterozygosity in heterologous alleles of the cfDNA in the second tissue sample relative to the cfDNA in the first tissue sample indicates the a decrease or absence in contaminating circulating tumor (ct) DNA (ctDNA) and therefore the treatment regimen is efficacious.

13. The method of assessing the efficacy of a treatment regimen of claim 1, wherein the tissue sample comprises a liquid biopsy.

14. The method of assessing the efficacy of a treatment regimen of claim 2, wherein the tissue obtained in the liquid biopsy comprises whole blood, plasma, serum, saliva, sputum, cerebral spinal fluid, urine, or lymph.

15. The method of assessing the efficacy of a treatment regimen of claim 1, wherein the cfDNA in the first or second tissue sample comprises circulating tumor (ct) DNA (ctDNA).

16. The method of assessing the efficacy of a treatment regimen of claim 1, wherein chromosomal homozygosity is measured using next generation sequencing (NGS), allelic-specific hybridization, primer extension, oligonucleotide ligation, and/or invasive cleavage.

17. The method of assessing the efficacy of a treatment regimen of claim 1, further comprising administering to the subject an anti-cancer treatment when a recurrent cancer and/or metastasis is detected.
